SEQUENCE LISTING (4 sequences)

SEQ ID NO: 1 - DNA, 4267 bp, Homo sapiens
ctccgaggaggggtggggacggtcctgacggtggggacatcaggctgccccgcagtaccagggagcgacttgaagtgcccatgccgcttgctccgggagaagcccaggtgtggcctcactcacatcccactctgagtccaaatgttctctccctggaagatatcaatgtttctgtctgttcgtgaggactccgtgcccaccacggcctctttcagcgccgacatgctcaatgtcaccttgcaagggcccactcttaacgggacctttgcccagagcaaatgcccccaagtggagtggctgggctggctcaacaccatccagccccccttcctctgggtgctgttcgtgctggccaccctagagaacatctttgtcctcagcgtcttctgcctgcacaagagcagctgcacggtggcagagatctacctggggaacctggccgcagcagacctgatcctggcctgcgggctgcccttctgggccatcaccatctccaacaacttcgactggctctttggggagacgctctgccgcgtggtgaatgccattatctccatgaacctgtacagcagcatctgtttcctgatgctggtgagcatcgaccgctacctggccctggtgaaaaccatgtccatgggccggatgcgcggcgtgcgctgggccaagctctacagcttggtgatctgggggtgtacgctgctcctgagctcacccatgctggtgttccggaccatgaaggagtacagcgatgagggccacaacgtcaccgcttgtgtcatcagctacccatccctcatctgggaagtgttcaccaacatgctcctgaatgtcgtgggcttcctgctgcccctgagtgtcatcaccttctgcacgatgcagatcatgcaggtgctgcggaacaacgagatgcagaagttcaaggagatccagacggagaggagggccacggtgctagtcctggttgtgctgctgctattcatcatctgctggctgcccttccagatcagcaccttcctggatacgctgcatcgcctcggcatcctctccagctgccaggacgagcgcatcatcgatgtaatcacacagatcgcctccttcatggcctacagcaacagctgcctcaacccactggtgtacgtgatcgtgggcaagcgcttccgaaagaagtcttgggaggtgtaccagggagtgtgccagaaagggggctgcaggtcagaacccattcagatggagaactccatgggcacactgcggacctccatctccgtggaacgccagattcacaaactgcaggactgggcagggagcagacagtgagcaaacgccagcagggctgctgtgaatttgtgtaaggattgagggacagttgcttttcagcatgggcccaggaatgccaaggagacatctatgcacgaccttgggaaatgagttgatgtctccggtaaaacaccggagactaattcctgccctgcccaattttgcagggagcatggctgtgaggatggggtgaactcacgcacagccaaggactccaaaatcacaacagcattactgttcttatttgctgccacacctgagccagcctgctccttcccaggagtggaggaggcctgggggcagggagaggagtgactgagcttccctcccgtgtgttctccgtccctgccccagcaagacaacttagatctccaggagaactgccatccagctttggtgcaatggctgagtgcacaagtgagttgttgccctgggtttctttaatctattcagctagaactttgaaggacaatttcttgcattaataaaggttaagccctgaggggtccctgataacaacctggagaccaggattttatggctcccctcactgatggacaaggaggtctgtgccaaagaagaatccaataagcacatattgagcacttgctgtatatgcagtattgagcactgtaggcaagagggaagaaagagaaggagccatctccatcttgaaggaactcaaagactcaagtgggaacgactgggcactgccaccaccagaaagctgttcgacgagacggtcgagcagggtgctgtgggtgatatggacagcagaagggggagaccaaggttccagctcaaccaataactattgcacaaccacctgtccctgcctcagttccctcttctgtaacatgaagtcgttgtgagggttaaaggcagtaacaggtataaagtacttagaaaagcaaagggtgctacgtacatgtgaggcatcattacgcagacgtaactgggatatgtttactataaggaaaagacactgaggtctagaaatagctccgtggagcagaatcagtattgggagccggtggcggtgtgaagcaccagtgtctggcacacagtaggtgctcattggctcccttccacctgtcattcccaccaccctgaggccccaaccgccacacacacaggagcatttggagagaaggccatgtcttcaaagtctgatttgtgatgaggcagaggaagatatttctaatcggtcttgcccagaggatcacagtgctgagaccccccaccaccagccggtacctgggaagggggagagtgcaggcctgctcagggactgttcctgtctcagcaaccaagggattgttcctgtcaatcaatggtttattggaaggtggcccagtatgagccctagaagagtgtgaaaaggaatggcaatggtgttcaccatcggcagtgccagggcagcactcattcacttgataaatgaatatttattagctggttggagagctagaacctggagaggctagaacctggagaactagaacctggagggctagaacctggagaggctagaaccaagaagggctagaacctggaggggctagaacctagagaagctaaaacctgagctagaagctggaggactagaacctggagggctggaatctggagagctagaacctggagggctagaacctggagggctagaatctggagagctagaacctggagggctagaatctggagagctagaacctggagggctagaacctggagagctagaacctagaagggctagaacctggagggctggaatctggagagctagaacctggagggctagaacctggagggctagaacctagaagggctagaacctggagggctggaatctggagagctagaacctggagggctagaacctggagggctagaacctagaagggctagaacctggagggctagaacctggcaggttagaacctagaagggctagaacctggagagccagaacctggagggctagaacctggaagggctagaacctgtagagctagaacatggagagctagaacccggcaggctagaacctggcaagctagaacctggagggaatgaacctggagggctagaacctggagaatgagaaaaatttacatggcaaagagcccataaatcctgaccaatccaactctgaattttaaagcaaaagcgtcaaaaaaaagattccctccttacccccaacccactcttttttcccaccacccactctcctctgcctcagtaagtatctggaggaagaaaacaggtgaaagaagaagtaaaaaccatttagtattagtattagaatgaagtcaaactgtgccacacatggtgaatgaaaaaaaaaaaaagaggctgtgttttgtcacacagggcagtcattcagcaccagagcacgtgatggtctgagactctcttaggagcagagctctgccgcaatggccatgtggggatccacacctggtctgaggggcaactgagtctgcgggagaagagcggccctatgcatggtgtagatgccctgataaagaacatctgtcctgtgaaagactcaatgagctgttatgttgtaaacaggaagcatttcacatccaaacgagaaaatcatgtaaacatgtgtcttttctgtagagcataataaatggatgaggtttttgcatagctctagcatttgttacaactcccgaaacccccgagtttggtccctggggtaccgccttgcacactcagaagcctttgggaaggggtgctattcatttctgctcaatctgttaacaggcttctggcatgtagatcagtggtctccaagcttttgtgattgtatattcctataggaaaaaaagaattgattatgcatacccagtatgtatacttattaatctgtatgaagatgtacattctaaaatataatcaaccagtagaaatttaagaaagaagatgtaaaaaa

SEQ ID NO: 2 - DNA, 1080 bp, Homo sapiens
catcccactctgagtccaaatgttctctccctggaagatatcaatgtttctgtctgttcgtgaggactccgtgcccaccacggcctctttcagcgccgacatgctcaatgccaccctagagaacatctttgtcctcagcgtcttctgcctgcacaagagcagctgcacggtggcagagatctacctggggaacctggccgcagcagacctgatcctggcctgcgggctgcccttctgggccatcaccatctccaacaacttcgactggctctttggggagacgctctgccgcgtggtgaatgccattatctccatgaacctgtacagcagcatctgtttcctgatgctggtgagcatcgaccgctacctggccctggtgaaaaccatgtccatgggccggatgcgcggcgtgcgctgggccaagctctacagcttggtgatctgggggtgtacgctgctcctgagctcacccatgctggtgttccggaccatgaaggagtacagcgatgagggccacaacgtcaccgcttgtgtcatcagctacccatccctcatctgggaagtgttcaccaacatgctcctgaatgtcgtgggcttcctgctgcccctgagtgtcatcaccttctgcacgatgcagatcatgcaggtgctgcggaacaacgagatgcagaagttcaaggagatccagacagagaggagggccacggtgctagtcctggttgtgctgctgctattcatcatctgctggctgcccttccagatcagcaccttcctggatacgctgcatcgcctcggcatcctctccagctgccaggacgagcgcatcatcgatgtaatcacacagatcgcctccttcatggcctacagcaacagctgcctcaacccactggtgtacgtgatcgtgggcaagcgcttccgaaagaagtcttgggaggtgtaccagggagtgtgccagaaagggggctgcaggtcagaacccattcagatggagaactccatgggcacactgcggacctccatctccgtggaacgccagattcacaaactgcaggactgggcagggagcagacagtgagcaaa

SEQ ID NO: 3 - protein, 391 aa, Homo sapiens
MFSPWKISMFLSVREDSVPTTASFSADMLNVTLQGPTLNGTFAQSKCPQVEWLGWLNTIQPPFLWVLFVLATLENIFVLSVFCLHKSSCTVAEIYLGNLAAADLILACGLPFWAITISNNFDWLFGETLCRVVNAIISMNLYSSICFLMLVSIDRYLALVKTMSMGRMRGVRWAKLYSLVIWGCTLLLSSPMLVFRTMKEYSDEGHNVTACVISYPSLIWEVFTNMLLNVVGFLLPLSVITFCTMQIMQVLRNNEMQKFKEIQTERRATVLVLVVLLLFIICWLPFQISTFLDTLHRLGILSSCQDERIIDVITQIASFMAYSNSCLNPLVYVIVGKRFRKKSWEVYQGVCQKGGCRSEPIQMENSMGTLRTSISVERQIHKLQDWAGSRQ

SEQ ID NO: 4 - protein, 351 aa, Homo sapiens
MFSPWKISMFLSVREDSVPTTASFSADMLNATLENIFVLSVFCLHKSSCTVAEIYLGNLAAADLILACGLPFWAITISNNFDWLFGETLCRVVNAIISMNLYSSICFLMLVSIDRYLALVKTMSMGRMRGVRWAKLYSLVIWGCTLLLSSPMLVFRTMKEYSDEGHNVTACVISYPSLIWEVFTNMLLNVVGFLLPLSVITFCTMQIMQVLRNNEMQKFKEIQTERRATVLVLVVLLLFIICWLPFQISTFLDTLHRLGILSSCQDERIIDVITQIASFMAYSNSCLNPLVYVIVGKRFRKKSWEVYQGVCQKGGCRSEPIQMENSMGTLRTSISVERQIHKLQDWAGSRQ

(19) United States
(12) Patent Application Publication
(10) Pub. No.: US 2009/0246269 A1
(43) Pub. Date: Oct. 1, 2009

(54) IDENTIFICATION AND USE OF GPRC VARIANTS IN THE TREATMENT AND DIAGNOSIS OF PARKINSON'S DISEASE

(76) Inventors: Vinayaka Kotraiah, Germantown, MD (US); Dehe Kong, Walkersville, MD (US); Matthew Paul Pando, Arlington, VA (US)

Correspondence Address: NIXON & VANDERHYE, PC, 901 NORTH GLEBE ROAD, 11TH FLOOR, ARLINGTON, VA, 22203, US

(21) Appl. No.: 12/308,710

(22) PCT Filed: Jun. 28, 2007
(86) PCT No.: PCT/EP2007/056504

§ 371 Date: Dec. 22, 2008

Related U.S. Application Data
(60) Provisional application No. 60/817,082, filed on Jun. 29, 2006.

(51) Int. Cl. A61K 39/395; C07H 21/04; C07K 14/435; C12Q 1/68; A61K 9/127; A61P 25/16

(52) U.S. Cl. 424/450; 536/23.5; 536/24.33; 530/350; 435/6; 424/141.1; 977/906

(57) ABSTRACT

The invention relates genes that are deregulated in Parkinson's disease tissues and the corresponding proteins are identified. These genes and the corresponding proteins are suitable targets for the treatment of Parkinson's disease. Also, the invention relates to compounds and their uses, particularly in the pharmaceutical industry. The invention more specifically relates to new uses of compounds that activate the B2 bradykinin receptor, for treating neurodegeneration involving oxidative stress and, more particularly, Parkinson's disease. The invention also relates to corresponding methods of treatment, and can be used in human subjects for preventive or curative treatment, either alone or in combination with other active agents or treatments.

### FIELD OF THE INVENTION

The present invention relates to the identification of DNA sequences that correspond to alternatively spliced isoforms of a gene expressed in Parkinson's disease. These isoforms or their corresponding proteins and the pathways they control are to be targeted for the treatment, prevention and/or diagnosis of neurodegenerative disease wherein these genes are differentially regulated and/or spliced, particularly in Parkinson's disease. The invention also relates to corresponding methods of treatment, and can be used in human subjects for preventive or curative treatment, either alone or in combination with other active agents or treatments.

### BACKGROUND OF THE INVENTION

Parkinson's disease (PD) is a progressive neurodegenerative disorder primarily characterized by muscular rigidity, tremor and abnormalities of posture. This emphasis on the motor disorder has overshadowed the cognitive and behavioral consequences of this disease. For instance, PD symptoms include a high incidence of depression and anxiety, and as many as 30% of all PD patients will experience dementia (Louis et al. 2004; Anderson 2004).

The pathological hallmark of PD is the degeneration of dopaminergic neurons of the subnucleus pars compactus of the substantia nigra. The classical movement disorders associated with Parkinson's disease begin to manifest when approximately 50% of the dopaminergic substantia nigra neurons have been lost. However, the neuronal loss is more widespread and affects other area of the brain, like the prefrontal cortex, which accounts for the non motor symptoms (Olanow and Tatton 1999).

Oxidative stress is the central phenomenon leading to neuronal death in PD (Tabner et al. 2001). This is not completely surprising due to the fact that the brain uses more oxygen and produces more energy per unit mass than any other organ, has a high iron content that can catalyze oxidation, and does not have a robust antioxidant enzyme defense (Kidd 2005). Compounding the brains susceptibility to oxidative degeneration is the inferior antioxidant defense mechanisms employed by mitochondria. It has been estimated that mitochondrial DNA is 10-100 times more likely to sustain damage than nuclear DNA (Floyd and Hensley 2002).

Current research supports the idea that mitochondrial disfunction is a common contributor to neurodegenerative disease. In PD, two genetically linked genes, DJ1 and PINK, are thought to be involved in protecting against mitochondrial damage/oxidative stress and mitochondrial homeostasis respectively (Kidd 2005). Impairment of mitochondrial complex I also occurs at a high rate in PD patients (Kidd 2000). It is also interesting to point out that all of the major toxins (MPTP, 6-OHDA, and rotenone) used to generate in vitro and in vivo models of PD primarily target mitochondrial complex I. Treatments with these compounds recapitulate many of the molecular and phenotypic alterations observed in PD. Regulation of the constant calcium flux that takes place in neurons also relies on a properly functioning mitochondrial, to maintain calcium homeostasis and avoid pushing the calcium equilibrium towards cell death (Toescu and Verkhratsky 2003).

The B₂ bradykinin receptor (BDKRB2) is a G-protein coupled receptor (GPCR) and is primarily expressed in neurons and smooth muscle cells (Perkins and Kelly 1993; Regoli et al. 1978; deBolis et al. 1989). The peptide hormone bradykinin binds to the BDKRB2 and primarily facilitates vasodilation. When the BDKRB2 is engaged by ligand it activates phospholipase C and phospholipase A₂, resulting in intracellular calcium mobilization (Burch and Axelrod 1987; Kaya et al. 1989; Slivka and Insel 1988), and specifically, mitochondrial calcium uptake (Visch et al. 2004). It has been demonstrated that bradykinin induced mitochondrial calcium accumulation and subsequent ATP synthesis is impaired in cells harboring complex I deficiencies and that normal ATP levels can be restored by facilitating an increase in mitochondrial calcium levels (Visch et al. 2004). Interestingly, only a 20% reduction in bradykinin-induced mitochondrial calcium uptake resulted in a 60% reduction in ATP production (Visch et al. 2004) and a 40% reduction in calcium uptake completely abolishes ATP production (Jouaville et al. 1999).

### SUMMARY OF THE INVENTION

The present invention relates to the identification of novel nucleic acid and amino acid sequences that are characteristic of Parkinson's disease, and which represent targets for therapy and/or diagnosis of such a condition in a subject.

The invention more specifically discloses specific isolated nucleic acid molecules that encode peptide sequences of novel alternative isoforms. These sequences were found to be differentially expressed between normal and Parkinson's disease tissue. These sequences and molecules represent targets and valuable information to develop methods and materials for the treatment of Parkinson's disease (“PD”).

It is an object of the invention to provide methods and materials for treatment of Parkinson's disease.

It is a more specific object of the invention to identify novel isoforms (novel splice variants) that are deregulated in Parkinson's disease tissue, which are potential gene targets for treatment of Parkinson's disease.

It is another specific object of the invention to identify exons and the corresponding protein domain encoded by those exons specifically deregulated in Parkinson's disease related cells.

It is another object of the invention to identify genes that are expressed in altered forms in PD tissue. These forms represent splice variants of the gene, where the SpliceArray™ detection probes either 1) indicates the splice event occurring within the gene, or 2) points to a gene that is actively spliced to produce different gene products. These different splice variants or isoforms can be targets for therapeutic intervention.

A particular object of this invention resides in a nucleic acid molecule selected from the group consisting of:
(i) a nucleic acid comprising the sequence contained in SEQ ID NO: 1 or 2;
(ii) variants of (i), wherein such variants comprise a nucleic acid sequence that is at least 70% identical to the sequence of (i) when aligned without allowing for gaps; and
(iii) fragments of (i) or (ii) having a size of at least 20 nucleotides in length.

Another specific object of this invention is a polypeptide comprising the amino acid sequence of SEQ ID NO: 3 or 4 or a fragment thereof.

It is another specific object of the invention to provide novel therapeutic regimens for the treatment of Parkinson's disease that involves the administration or use of ligands, peptides or small molecules, alone or in combination with other active agents or treatments.

In this regard, a particular object of this invention resides in the use of a B₂ bradykinin receptor (BDKRB2) agonist, particularly an antibody, a peptide or a small molecule agonist, for the manufacture of a medication for treating Parkinson's disease.

A further object of this invention resides in the use of a BDKRB2 agonist for the manufacture of a medicament for protecting neurons from oxidative stress in subjects having Parkinson's Disease.

Another object of this invention resides in the use of a BDKRB2 agonist for the manufacture of a medicament for protecting dopaminergic neurons in a subject having Parkinson's Disease.

Another aspect of this invention is a method of treating Parkinson's Disease, comprising administering to a subject in need thereof an effective amount of a BDKRB2 agonist.

A further object of this invention is a method for treating Parkinson's disease, which comprises administering to a subject a therapeutically effective amount of a ligand, which specifically binds a target molecule selected from a nucleic acid molecule of claim 1 or 2 and a polypeptide of claim 4.

It is another object of this invention to provide pharmaceutical compositions comprising an agonist as defined above, in combination with a pharmaceutically acceptable carrier or excipient.

The invention also relates to a primer mixture that comprises primers that result in the specific amplification of one of the nucleic acid sequences as defined in the present application.

The invention also relates to a diagnostic kit for detection of Parkinson's disease which comprises a nucleic acid as defined above and a detectable label.

The invention can be used in human subjects for preventive or curative treatment, either alone or in combination with other active agents or treatments.

### LEGEND TO THE FIGURES

FIG. 1: TMHMM analysis of BDKRB2 reference form depicting extracellular (pink), transmembrane (red), and intracellular regions of the protein (blue). The protein exhibits the classical profile of a seven transmembrane spanning G-protein coupled receptor.

FIG. 2: TMHMM analysis of BDKRB2 variant form depicting extracellular (pink), transmembrane (red), and intracellular regions of the protein (blue). As compared to the profile of the reference form, depicted in FIG. 1, the N-terminus is now predicted to be intracellular and the first transmembrane domain appears to be disrupted. The deletion event contained in the variant isoform is likely to have profound effects on ligand binding and receptor activation.

FIG. 3: Log ratio of the relative quantity (RQ) of BDKRB2 expression for substantia nigra-putamen pools from Parkinson's verses normal patients, substantia nigra from Parkinson's verses normal patients, and putamen from Parkinson's verses normal patients. BDKRB2 is significantly down regulated in the Parkinson's patient substantia nigra-putamen pool, the Parkinson's substantia nigra, but not the Parkinson's putamen. This demonstrates that BDKRB2 is specifically and significantly down regulated in the substantia nigra of Parkinson's patients.

FIG. 4: Log ratio of the relative quantity (RQ) of BDKRB2 expression for neuroblastoma cells treated with 100 nM rotenone or solvent. BDKRB2 is significantly down regulated at the 24 hour time-point in this acute model of oxidative stress.

FIG. 5: Log ratio of the relative quantity (RQ) of BDKRB2 expression for neuroblastoma cells treated with 5 nM rotenone or solvent. BDKRB2 is initially slightly up regulated at 1 week of treatment but is then significantly down regulated at the 2 week time-point in this chronic model of oxidative stress.

FIG. 6: Western blot with an anti-BDKRB2 antibody showing that while RNA levels of BDKRB2 decrease after 24 hours of rotenone treatment protein levels do not decrease until the 48 hour time-point. GAPDH is used as a loading control.

FIG. 7: BDKRB2 reference form expression in the striatum of mouse MPTP model. Log ratios of the relative quantity (RQ) of BDKRB2 expression in the MPTP treated striatum compared to the saline treated striatum are plotted. BDKRB2 is significantly down regulated at the 3 and 7 day time-points in this animal model of Parkinson's disease. Mice were treated with either MPTP (45 mg/kg) or saline and striatal tissues were extracted at 1 day, 3 day, 7 days and 14 days post MPTP injection.

FIG. 8: RT-PCR performed on RNA from patient samples and nueroblastoma cells treated with 100 nM rotenone or solvent. The primers used flank the partial internal exon deletion event present in the BDKRB2 variant and amplify a section of both the reference (upper band) and variant (lower band). Both isoforms are present in normal and Parkinson's substantia nigra and putamen tissue. There may be a slightly higher ratio of the variant form in the Parkinson's samples but this needs to be more closely quantified. In the acute rotenone treated samples, it appears that by 48 hour of treatment the reference form is down regulated and the variant form is up regulated resulting in an isoform ratio shift. The observed isoform/ratio shift is likely to decrease the level of signaling coming from the B₂ bradykinin receptors.

FIG. 9: Effect of bradykinin titration on the activity of BDKRB2 reference form. CHO-K1 cells were transfected with pNFAT—Firefly Luciferase reporter, BDKBR2-pcDNA3.1 and pGL4.73 [hRenilla Luciferase/SV40]. 18 hrs after transfection, cells were treated with bradykinin at indicated concentrations in 0.05% FBS medium for 1, 4, 8 and 24 hrs. The Firefly luciferase reporter activity and the renilla luciferase activity were measured sequentially. BDKRB2 activity was estimated by calculating the Ratios of Firefly and Renilla Luciferase Units (RLU). A significant increase in RLU was observed after 4 and 8 hours of treatment with bradykinin.

FIG. 10: Effect of bradyzide titration on the activity of BDKRB2 reference form. CHO-K1 cells were transfected with pNFAT—Firefly Luciferase reporter, BDKBR2-pcDNA3.1 and pGL4.73 [hRenilla Luciferase/SV40]. 18 hrs after transfection, cells were treated with bradyzide at indicated concentration in 10% FBS medium for 8, 24 and 48 hrs. The Firefly luciferase reporter activity and the renilla luciferase activity were measured sequentially. BDKRB2 activity was estimated by calculating the Ratios of Firefly and Renilla Luciferase Units (RLU). Significant serum-induced basal BDKRB2 activity was observed at the 8 hour time-point and this was reduced by about 40% through the addition of bradyzide.

FIG. 11: Signaling activities of the BDKRB2 reference form and BDKRB2 variant. CHO-K1 cells were transfected with pNFAT—Firefly Luciferase reporter, pGL4.73 [hRenilla Luciferase/SV40] and different amounts of either BDKBR2-pcDNA3.1 or BDKRB2 variant—pcDNA3.1 plasmids. 18 hrs after transfection, cells were treated with 200 nM of bradykinin in media containing 0.05% FBS for 8 hrs. The Firefly luciferase reporter activity and the Renilla luciferase activity were measured sequentially. BDKRB2 activity was estimated by calculating the Ratios of Firefly and Renilla Luciferase Units (RLU). The RLU in cells transfected with BDKRB2 variant were not significantly different from RLU in untransfected cells. Whereas, the RLU in cells transfected with the BDKRB2 reference form increased significantly up to 30 ng of plasmid and then started to decrease, suggesting that the system can get saturated with increased expression of the receptor.

FIG. 12: Effect of the BDKRB2 variant on signaling mediated by BDKRB2 reference form. CHO-K1 cells were transfected with pNFAT—Firefly Luciferase reporter (0.9 ug), pGL4.73 [hRenilla Luciferase/SV40] (18 ng), BDKBR2-pcDNA3.1 (15 ng) and BDKBR2-variant-pcDNA3.1 (from 0 to 750 ng) for 24 hrs in 10% FBS medium. The Firefly luciferase reporter activity and the Renilla luciferase activity were measured sequentially. BDKRB2 activity was estimated by calculating the Ratios of Firefly and Renilla Luciferase Units (RLU). The signaling activity in the BDKRB2 transfected cells is presumably due to the bradykinin present in serum. This activity was inhibited in a dose-dependent manner by increasing amounts of transfected BDKRB2 variant.

FIG. 13: Effect of the BDKRB2 variant on signaling mediated by the BDKRB2 reference form. CHO-K1 cells were transfected with pNFAT—Firefly Luciferase reporter, pGL4.73 [hRenilla Luciferase/SV40] and BDKBR2-pcDNA3.1 and/or BDKBR2-variant-pcDNA3.1. 18 hrs after transfection, cells were treated with bradykinin (0 to 10 uM) in media containing 0.05% FBS for 8 hrs. The Firefly luciferase reporter activity and the Renilla luciferase activity were measured sequentially. BDKRB2 activity was estimated by calculating the Ratios of Firefly and Renilla Luciferase Units (RLU). The inhibitory effect of the variant on basal and ligand dependent BDKRB2 signaling activity is not overcome in the presence of increasing bradykinin concentrations.

FIG. 14: Effect of bradykinin on rotenone toxicity in SH-SY5Y cells. SH-SY5Y cells were pretreated with bradykinin (0 to 10 uM) in medium containing 10% FBS for 4 hours, then treated with 100 nM rotenone for 40 hours. The medium was replaced with 0.05% FBS medium without changing the bradykinin and rotenone concentrations for an additional 8 hrs. ATP levels were measured using a luminescence-based assay and the Relative Counts Per Second (CPS) were plotted against bradykinin concentration. An increase in ATP levels was observed in cells treated with bradykinin (300 nM and higher) over that observed in cells treated with rotenone alone.

### DETAILED DESCRIPTION OF THE INVENTION

As indicated above, the present invention relates to the identification of novel therapeutic targets and treatments, particularly the B₂ bradykinin receptor (BDKRB2) and its agonists, for treating Parkinson's Disease (PD).

SpliceArrays™ analyze structural differences between expressed gene transcripts and provides systematic access to alterations in RNA splicing (disclosed in U.S. Pat. No. 6,881,571 and EP1,062,364, the disclosure of which is incorporated by reference in its entirety). Having access to expression data for these alternative splice events, which are critical for cellular homeostasis, represents a useful advance in functional genomics and target discovery.

The present invention is based in part on the identification of deregulated exons that are identified using SpliceArrays™. Differential expression of given exons is determined by calculating the indirect log ratios of probe intensities obtained through indirect comparisons of selected normal, Parkinson's disease, and Universal RNA samples against hybridizing a microarray designed to monitor alternative splicing, which is known to those skilled in the art. Specifically, two alternative isoforms were identified through SpliceArray™ analysis and confirmed to be differentially expressed between normal substantia nigra and Parkinson's disease substantia nigra tissue. This alternative usage of exons in different biological samples produces different gene products from the same gene through a process well known in the art as alternative RNA splicing.

Alternatively spliced mRNA's produced from the same gene contain different ribonucleotide sequence, and therefore translate into proteins with different amino acid sequences. Nucleic acid sequences that are alternatively spliced into or out of the gene products can be inserted or deleted in frame or out of frame from the original gene sequence. This leads to the translation of different proteins from each variant. Differences can include simple sequence deletions, or novel sequence information inserted into the gene product. Sequences inserted out of frame can lead to the production of an early stop codon and produce a truncated form of the protein. Alternatively, in-frame insertions of nucleic acid may cause an additional protein domain to be expressed from the mRNA. The end stage target is a novel protein containing a novel epitope and/or function. Many variations of known genes have been identified and produce protein variants that can be agonistic or antagonistic with the original biological activity of the protein.

SpliceArray™ analysis thus identifies genes and proteins which are subject to differential regulation and alternative splicing(s) in Parkinson's disease. SpliceArray™ results thus allow the definition of target molecules suitable for therapy of Parkinson's disease and potentially other related neurodegenerative diseases, which target molecules comprise all or a portion of genes or RNAs monitored by the SpliceArray™, as well as corresponding polypeptides or proteins, and variants thereof.

A particular object of this invention thus resides in a target molecule selected from the group consisting of:
(i) a nucleic acid (preferably a DNA or RNA) comprising the sequence contained in SEQ ID NO: 1 or 2;
(ii) variants of (i), wherein such variants comprise a nucleic acid sequence that is at least 70% identical to the sequence of (i) when aligned without allowing for gaps; and
(iii) fragments of (i) or (ii) having a size of at least 20 nucleotides in length
(iv) polypeptides encoded by a nucleic acid of any one of (i) to (iii).

A first type of target molecule is a target nucleic acid molecule comprising the sequence of a full gene or RNA molecule comprising the events monitored by the SpliceArray™ and disclosed in the present application. Indeed, since SpliceArrays™ identify genetic deregulations associated with Parkinson's disease, the whole gene or RNA sequence from which said monitored event derives can be used as a target of therapeutic intervention.

Similarly, another type of target molecule is a target polypeptide molecule comprising the sequence of a full-length protein comprising the amino acid sequence encoded by a monitored event as disclosed in the present application.

These target molecules (including genes, fragments, proteins and their variants) can serve as targets for the development of therapeutics. For example, these therapeutics may modulate biological processes associated with neuronal cell viability. Agents may also be identified that are associated with the inhibition of apoptosis (cell death) in Parkinson's disease related neurons.

Specifically, the invention provides variant sequences that are expressed and are deregulated in Parkinson's disease. These sequences are from genes identified to be important in, impact or regulate neuronal cell viability.

As noted, the present invention provides novel splice variants of genes that correlate to Parkinson's disease. The present invention also embraces variants thereof. As used herein “variants” means sequences that are at least about 75% identical, more preferably at least about 85% identical, and most preferably at least 90% identical and still more preferably at least about 95-99% identical to a reference sequence. Such identity is typically measured by sequence alignment without allowing for gaps. The term “variants” also encompasses nucleic acid sequences that hybridize to the subject sequence under high, moderate or low stringency conditions e.g., as described infra. Typical stringent hybridisation conditions include temperatures above 30° C., preferably above 35° C., more preferably in excess of 42° C., and/or salinity of less than about 500 mM, preferably less than 200 mM. Hybridization conditions may be adjusted by the skilled person by modifying the temperature, salinity and/or the concentration of other reagents such as SDS, SSC, etc.

Also, the present invention provides for primer pairs that result in the amplification of DNAs encoding the subject novel genes or a portion thereof in an mRNA library obtained from a desired cell source, typically human neuronal cells or Parkinson's disease tissue samples. Typically, such primers will be on the order of 12 to 100 nucleotides in length, and will be constructed such that they provide for amplification of the entire or most of the target gene.

“Variant protein” refers to a protein possessing an amino acid sequence that possess at least 90% sequence identity, more preferably at least 91% sequence identity, even more preferably at least 92% sequence identity, still more preferably at least 93% sequence identity, still more preferably at least 94% sequence identity, even more preferably at least 95% sequence identity, still more preferably at least 96% sequence identity, even more preferably at least 97% sequence identity, still more preferably at least 98% sequence identity, and most preferably at least 99% sequence identity, to the corresponding native human amino acid sequence wherein sequence identity is as defined infra. Preferably, this variant will possess at least one biological property in common with the native protein.

“Fragment of encoding nucleic acid molecule or sequence” refers to a nucleic acid sequence corresponding to a portion of the reference molecule or sequence, wherein said portion is at least about 50 nucleotides in length, or 100, more preferably at least 150 nucleotides in length. A fragment is most preferably a distinctive fragment, i.e., comprises a junction sequence caused by splicing.

Based on the results contained in this application, it is proposed that the disclosed genes that are associated with the differentially expressed sequences and the corresponding variant proteins represent suitable targets for Parkinson's disease therapy or prevention, e.g. for the development of antibodies or peptide ligands and small molecular agonists. The potential therapies are described in greater detail below.

Based on the identification of splicing alterations identified by our SpliceArray™ analysis of PD patient samples versus normal patient samples, several unprecedented pathways, receptors and enzymes were identified.

One receptor identified by this analysis is the B₂ bradykinin receptor (BDKRB2). Our identification of differentially regulated splicing and down regulation of BDKRB2 in Parkinson's related samples is the first direct link between bradykinin signaling and PD. Activating bradykinin signaling and specifically those signaling pathways controlled by the BDKRB2 represent a new therapeutic approach to rescue and protect dopaminergic neurons from oxidative stress and energy depletion, and more precisely from the oxidative stress induced neurotoxicity observed in a disease like Parkinson's disease.

The BDKRB2 is a G-protein coupled receptor (GPCR) and is primarily expressed in neurons and smooth muscle cells (Perkins and Kelly 1993; Regoli et al. 1978; deBolis et al. 1989). The peptide hormone bradykinin binds to the BDKRB2 and primarily facilitates vasodilation. When the BDKRB2 is engaged by ligand it activates phospholipase C and phospholipase A₂, resulting in intracellular calcium mobilization (Burch and Axelrod 1987; Kaya et al. 1989; Slivka and Insel 1988), and specifically, mitochondrial calcium uptake (Visch et al. 2004). It has been demonstrated that bradykinin induced mitochondrial calcium accumulation and subsequent ATP synthesis is impaired in cells harboring complex I deficiencies and that normal ATP levels can be restored by facilitating an increase in mitochondrial calcium levels (Visch et al. 2004). Decreased mitochondrial function leads directly to increased oxidative stress and decreases cell viability. Interestingly, only a 20% reduction in bradykinin-induced mitochondrial calcium uptake resulted in a 60% reduction in ATP production (Visch et al. 2004) and a 40% reduction in calcium uptake completely abolishes ATP production (Jouaville et al. 1999).

The present invention now demonstrated that BDKRB2 agonists can be used in the treatment of Parkinson's disease.

### BDKRB2 Agonists

Within the context of this invention, a BDKRB2 agonist designates any peptide, compound, agent or treatment that activates (e.g., increases or stimulates) BDKRB2 or its variants, more preferably that activate BDKRB2-controlled intracellular signaling pathway(s). Such agonists include more specifically any compound that stimulates the BDKRB2.

In a preferred embodiment, the agonists have an IC50 for BDKRB2 which is below 1 mM and, more preferably, below 50 nM.

Furthermore, preferred BDKRB2 agonists can get through (i.e., cross) the blood-brain barrier (BBB). In this regard, the agonists to be used in the present invention generally present a molecular weight less than about 800 daltons, preferably less than about 600 daltons.

Preferred BDKRB2 agonists are selective activators, i.e., they are essentially active on BDKRB2 with no substantial and direct specific activity on other receptors.

Other agonists to be used in this invention are BDKRB agonists, i.e., they are capable of activating various subtypes of BDKRB receptors, such as BDKRB1 and/or BDKRB2.

In a particular embodiment, the agonists can activate either BDKRB2 or BDKRB1, or both (i.e., dual activators). Alternatively, a combination comprising a BDKRB2 agonist and a BDKRB1 agonist can be used.

Example BDKRB2 agonists for use in the present invention are listed below
Bradykinin: Arg-Pro-Pro-Gly-Phe-Ser-Pro-Phe-Arg-OH;
[Hyp3]-Bradykinin: Arg-Pro-Hyp-Gly-Phe-Ser-Pro-Phe-Arg (Kato et al. 1988);
FR 190997 (Rizzi et al. 1999); and
Labradimil (Emerich et al. 2001).

The present invention also includes, as BDKRB2 agonists, the optical and geometrical isomers, racemates, tautomers, salts, hydrates and mixtures of the above cited compounds.

Also, it should be understood that the present invention is not limited to the compounds identified above, but shall also include any compound and derivative thereof cited in the references mentioned above, as well as all BDKRB2 agonists known to the man skilled in the art, which are appropriate for use in human subjects.

Also, other types of agonists include antibodies (or derivatives or fragments thereof) which bind a BDKRB2 receptor and activate the same. Antibodies may be either polyclonal or, preferably, monoclonal. Also, antibody derivatives include any molecule derived from an antibody, which exhibit at least substantially the same antigen specificity, such as human antibodies, humanized antibodies, single chain antibodies, etc. Antibody fragments include Fab, Fab′2, CDR, etc.

Other possible agonists include specific peptides that bind a BDKRB2 and activate at least one signaling pathway.

The activity of an agonist can be verified by assays known per se in the art, such as a binding assay (e.g., in vitro or in a cell-based system) and/or a functional assay, to measure cell signalling pathways (calcium release, ATP synthesis, etc.).

Furthermore, the BDKRB2 agonists also include pro-drugs of compounds cited above which, after administration to a subject, are converted to said compounds. They also include metabolites of compounds cited above which display similar therapeutic activity to said compounds.

### Formulation and Administration

The BDKRB2 agonist according to the invention may be formulated in any appropriate medium or formulation or composition suitable for use in human subjects. Typically, such formulations or compositions include pharmaceutically acceptable carrier(s) or excipient(s), such as isotonic solutions, buffers, saline solution, etc. The formulations may include stabilizers, slow-release systems, surfactants, sweeteners, etc. Such formulations may be designed for various administration routes, including systemic injection (e.g., intravenous, intracerebral, intramucular, transdermic, etc.) or oral administration.

The compositions may contain physiologically acceptable diluents, fillers, lubricants, excipients, solvents, binders, stabilizers, and the like. Diluents that may be used in the compositions include but are not limited to dicalcium phosphate, calcium sulphate, lactose, cellulose, kaolin, mannitol, sodium chloride, dry starch, powdered sugar and for prolonged release tablet-hydroxy propyl methyl cellulose (HPMC). The binders that may be used in the compositions include but are not limited to starch, gelatin and fillers such as sucrose, glucose, dextrose and lactose.

Natural and synthetic gums that may be used in the compositions include but are not limited to sodium alginate, ghatti gum, carboxymethyl cellulose, methyl cellulose, polyvinyl pyrrolidone and veegum. Excipients that may be used in the compositions include but are not limited to microcrystalline cellulose, calcium sulfate, dicalcium phosphate, starch, magnesium stearate, lactose, and sucrose. Stabilizers that may be used include but are not limited to polysaccharides such as acacia, agar, alginic acid, guar gum and tragacanth, amphotsics such as gelatin and synthetic and semi-synthetic polymers such as carbomer resins, cellulose ethers and carboxymethyl chitin.

Solvents that may be used include but are not limited to Ringers solution, water, distilled water, dimethyl sulfoxide to 50% in water, propylene glycol (neat or in water), phosphate buffered saline, balanced salt solution, glycol and other conventional fluids.

The compounds may be formulated in various forms, including solid and liquid forms, such as injectable solutions, capsules, tablets, gel, solution, syrup, suspension, powder, etc.

In a particular embodiment, the BDKRB2 agonists according to the invention are incorporated into a specific pharmaceutical formulation or technology that enables their delivery to the human brain using catalyzed-transport systems. Specific pharmaceutical formulations include, for instance, suitable liposomal carriers to encapsulate neuroactive compounds that are stable enough to carry them to the brain across the BBB with the appropriate surface characteristics for an effective targeting and for an active membrane transport.

Specific technologies include, for instance, suitable nanoparticle-based brain drug delivery systems to deliver drugs to the brain. These systems mask the BBB-limiting characteristics of the drug, enable targeted brain delivery via BBB transporters and provide a sustained release in brain tissue which could reduce dosage frequency, peripheral toxicity, and adverse effects.

Other suitable pharmaceutical formulations are disclosed in the prior art literature, such as in U.S. Pat. No. 5,874,442; WO01/46137; WO97/30992; WO98/00409 or WO97/17070, for instance, which are incorporated therein by reference.

Appropriate dosages and regimens may be determined by the skilled artisan, based on the present description and the available prior art literature. In particular, repeated administrations may be performed, with dosages ranging from 0.001 to 100 mg.

The invention allows effective treatment of Parkinson's Disease, e.g., a reduction in symptoms, disease progression, muscular rigidity or tremor. The treatment may be carried out using any such BDKRB2 agonist, either alone or in combination(s), optionally combined to other therapeutically active agents.

### Products and Diagnosis

As discussed above, the present invention discloses a novel target involved in neuro-protection. Furthermore, the invention show that genetic alterations occur within this gene, which represent valuable therapeutic targets, e.g., for drug screening or disease diagnosis, as well as for use as active agents or immunogens.

In this context, the invention particularly describes the appearance of alternative forms of the mRNA encoding BDKRB2 in neuronal cells from PD patients or subjected to oxidative stress, and particularly of forms altered within the coding sequence. Other forms can be envisioned and investigated within the scope of the present application.

Accordingly, the present invention relates to methods of detecting the presence or predisposition to oxidative stress comprising detecting, in a sample from a subject, the presence of an altered BDKRB2 locus, the presence of such altered locus being indicative of the presence or predisposition to oxidative stress.

A further object of this invention is a method of selecting drugs, comprising a step of determining whether a candidate drug can alter the BDKRB2 locus, e.g., the (relative) amount of splicing forms of said gene.

Within the context of the invention, the term BDKRB2 locus denotes any sequence or any BDKRB2 product in a cell or an organism. This term particularly means the nucleic acid sequences, either coding or non-coding, as well as the protein sequences, whether mature or not. Therefore, the term BDKRB2 locus includes all or part of the genomic DNA, including its coding and/or non-coding regions (introns, regulatory sequences, etc.), the RNA (messenger, pre-messenger, etc.) and the BDKRB2 proteins (precursor, mature, soluble, secreted, etc. forms), present in an organism, tissue or cell.

The term “BDKRB2 gene” denotes any nucleic acid encoding a BDKRB2 polypeptide. It can be genomic (gDNA), complementary (cDNA), synthetic or semi-synthetic DNA, mRNA, synthetic RNA, etc. It can be a recombinant or synthetic nucleic acid, produced by techniques known to those skilled in the art, such as artificial synthesis, amplification, enzymatic cleavage, ligation, recombination, etc., using biological sources, available sequences or commercial material. BDKRB2 gene exists typically in a two-stranded form, even though different forms can exist according to the invention. The sequence of the BDKRB2 gene is available in certain data banks, such as, notably, RefSeq, no NM_{—}000632. Other BDKRB2 gene sequences, according to the invention, can be isolated from samples, or collections, or may be synthesized. BDKRB2 sequences can relate to sequences that hybridize in highly stringent conditions with a nucleic acid encoding the sequences in SEQ ID NO: 1 and SEQ ID NO: 2 presented below.

The term BDKRB2 polypeptide particularly denotes any polypeptide encoded by a BDKRB2 gene as defined herein above. A specific example is supplied below (SEQ ID NO: 3), corresponding to the sequence referenced in Genbank under the number NP_{—}000614.

The term BDKRB2 polypeptide also includes, in the broad sense, any biologically active natural variant of the sequence identified above that could result from polymorphisms, splicing, mutations, insertions, etc.

Alteration of the BDKRB2 locus can be of a diverse nature, such as, in particular, one or several mutations, insertions, deletions and/or spicing events or the like, in the gene or RNA encoding BDKRB2. Advantageously it is a splicing event, for example the appearance of a splice form of BDKRB2 or modification of the ratio between different splice forms or between a non-spliced form and spliced forms.

In more preferred embodiments, the above methods comprise detecting the presence of an altered splicing of BDKRB2, e.g., the appearance of particular splicing isoforms or the presence of an altered ratio between splicing isoforms. More specifically, the method comprises detecting the presence or (relative) amount of a nucleic acid molecule comprising the sequence SEQ ID NO: 1 or 2, or of a polypeptide comprising the sequence of SEQ ID NO: 3 or 4, or a distinctive fragment thereof.

Such nucleic acid molecules and polypeptides also represent particular object of the present invention, as well as any distinctive fragment or analogs thereof; antibodies specifically binding to such polypeptides and specific nucleic acid probes or primers. In this regard, the invention relates to a polypeptide comprising SEQ ID NO: 4 or a distinctive fragment thereof, said distinctive fragment thereof comprising at least the sequence ADMLNATLEN, corresponding to residues 26-35 of SEQ ID NO: 4.

A particular object of this invention resides in methods for detecting the presence, stage or risk of PD in a subject, the method comprising detecting in vitro or ex vivo the presence of an altered BDKRB gene expression in a sample from the subject, the presence of such an altered BDKRB gene expression being indicative of the presence, stage or risk of a PD in said subject.

According to specific embodiments of the method, the altered expression is an increased expression of BDKRB splicing forms in said sample.

Another object of this invention resides in methods for detecting the presence, stage or risk of PD in a subject, the method comprising determining in vitro or ex vivo the (relative) amount of splicing isoform(s) of BDKRB2 gene or protein in a sample from the subject, such amount being indicative of the presence, stage or risk of a PD in said subject. In a particular embodiment, the amount determined is compared to a control or mean value, or to that measured in a control sample. In an other embodiment, the ratio of splicing isoform(s)/native isoform is determined, and any increase in such ratio is indicative of the presence of such a PD.

Another object of this invention resides in methods for detecting the presence, stage or risk of a PD in a subject, the method comprising determining in vitro or ex vivo the (relative) amount of a BDKRB2 RNA isoform comprising the sequence of SEQ ID NO: 1 or 2 or a distinctive fragment thereof in a fluid sample derived from the subject, preferably of a BDKRB2 RNA isoform comprising the sequence of SEQ ID NO: 2 or a distinctive fragment thereof. In a particular embodiment, the ratio of a BDKRB2 RNA isoform comprising the sequence of SEQ ID NO: 2/a BDKRB2 RNA isoform comprising the sequence of SEQ ID NO: 1 is determined, an increase in such a ratio being indicative of the presence, stage or risk of a PD in a subject.

Another object of this invention resides in methods for detecting the presence, stage or risk of a PD in a subject, the method comprising determining in vitro or ex vivo the (relative) amount of a BDKRB2 protein isoform comprising the sequence of SEQ ID NO: 3 or 4 or a distinctive fragment thereof in a fluid sample derived from the subject, preferably of a BDKRB2 protein isoform comprising the sequence of SEQ ID NO: 4 or a distinctive fragment thereof. In a particular embodiment, the ratio of a BDKRB2 protein isoform comprising the sequence of SEQ ID NO: 4/a BDKRB2 protein isoform comprising the sequence of SEQ ID NO: 2 is determined, an increase in such a ratio being indicative of the presence, stage or risk of a PD in a subject.

In a typical embodiment, the fluid sample derives (e.g., by dilution, concentration, purification, separation, etc.) from total blood, serum, plasma, urine, etc.

A further object of this invention is a method of assessing the efficacy of a treatment of PD in a subject, the method comprising comparing (in vitro or ex vivo) BDKRB2 gene expression in a sample from the subject prior to and after said treatment, an decreased expression of splicing isoforms being indicative of a positive response to treatment.

The invention further relates to methods of selecting biologically active compounds on PD, the method comprising a step of selecting compounds that mimic or stimulate BDKRB2 expression or activity.

In a specific embodiment, the method comprises contacting a test compound with a recombinant host cell comprising a reporter construct, said reporter construct comprising a reporter gene under the control of a BDKRB2 gene promoter, and selecting the test compounds that stimulate expression of the reporter gene.

A further aspect of this invention resides in a nucleic acid primer that allows (specific) amplification of (a) splicing isoform(s) of BDKRB2, or allows to discriminate between splicing and native isoforms, particularly between isoforms of BDKRB2 comprising SEQ ID NO: 1 or 2, respectively, or a distinctive fragment thereof.

A further aspect of this invention resides in a nucleic acid probe that (specifically) hybridizes to a splicing isoform of BDKRB2, or allows to discriminate between splicing and native isoforms, particularly between isoforms of BDKRB2 comprising SEQ ID NO: 1 or 2, respectively, or a distinctive fragment thereof.

A further aspect of this invention resides in an antibody (including derivatives thereof and producing hybridomas), that (specifically) binds a splicing isoform of a BDKRB2 protein or allows to discriminate between splicing and native isoforms, particularly between isoforms of BDKRB2 comprising SEQ ID NO: 3 or 4, respectively, or a distinctive fragment thereof. A particularly preferred antibody is an antibody (or a fragment or derivative thereof) that binds a polypeptide comprising a sequence ADMLNATLEN, corresponding to residues 26-35 of SEQ ID NO: 4, or that has been raised using an immunogen comprising residues 26-35 of SEQ ID NO: 4.

The invention further relates to kits comprising a primer, probe or antibody as defined above. Such kits may comprise a container or support, and/or reagents to perform an amplification, hybridization or binding reaction.

Various techniques known in the art may be used to detect or quantify altered BDKRB2 expression, including sequencing, hybridisation, amplification and/or binding to specific ligands (such as antibodies). Other suitable methods include allele-specific oligonucleotide (ASO), allele-specific amplification, Southern blot (for DNAs), Northern blot (for RNAs), single-stranded conformation analysis (SSCA), PFGE, fluorescent in situ hybridization (FISH), gel migration, clamped denaturing gel electrophoresis, heteroduplex analysis, RNase protection, chemical mismatch cleavage, ELISA, radio-immunoassays (RIA) and immuno-enzymatic assays (IEMA).

Amplification may be performed according to various techniques known in the art, such as by polymerase chain reaction (PCR), ligase chain reaction (LCR), strand displacement amplification (SDA) and nucleic acid sequence based amplification (NASBA). These techniques can be performed using commercially available reagents and protocols. Preferred techniques use allele-specific PCR or PCR-SSCP. Amplification usually requires the use of specific nucleic acid primers, to initiate the reaction.

Nucleic acid primers useful for amplifying sequences from the BDKRB2 gene or RNA are complementary to and specifically hybridize with a portion of the BDKRB2 gene or RNA. Most preferred primers for use in the present invention allow the amplification of (a) splicing isoform(s) of BDKRB2, e.g., contain a sequence that is complementary and specifically hybridizes to a junction sequence caused by splicing. A specific example of such junction is the sequence TCAATGCCACCC, corresponding to nucleotide residues 104-115 of SEQ ID NO: 2.

Typical primers of this invention are single-stranded nucleic acid molecules of about 5 to 60 nucleotides in length, more preferably of about 8 to about 25 nucleotides in length. The sequence can be derived directly from the sequence of the BDKRB2 gene. Perfect complementarity is preferred, to ensure high specificity. However, certain mismatch may be tolerated.

The invention also concerns the use of a nucleic acid primer or a pair of nucleic acid primers as described above in a method of detecting the presence of or predisposition to PD in a subject or in a method of assessing the response of a subject to a treatment of PD.

In another embodiment, detection is carried out by a technique using selective hybridization. A particular detection technique involves the use of a nucleic acid probe specific for BDKRB2 gene or RNA, followed by the detection of the presence and/or (relative) amount of a hybrid. The probe may be in suspension or immobilized on a substrate or support (as in nucleic acid array or chips technologies). The probe is typically labeled to facilitate detection of hybrids.

In this regard, a particular embodiment of this invention comprises contacting the sample from the subject with a nucleic acid probe specific for (a) splicing isoform(s) of BDKRB2, and assessing the formation of a hybrid. In a particular embodiment, the method comprises contacting simultaneously the sample with a set of probes that are specific, respectively, for splicing and native isoforms of BDKRB2.

Within the context of this invention, a probe refers to a polynucleotide sequence which is complementary to and capable of specific hybridisation with a (target portion of a) BDKRB2 gene or RNA, and which is suitable for detecting the presence or (relative) amount thereof in a sample. Probes are preferably perfectly complementary to a sequence of the BDKRB2 gene or RNA. Probes typically comprise single-stranded nucleic acids of between 8 to 1000 nucleotides in length, for instance of between 10 and 800, more preferably of between 15 and 700, typically of between 20 and 500. A specific example of a probe is specific and/or complementary to the sequence TCAATGCCACCC, corresponding to nucleotide residues 104-115 of SEQ ID NO: 2.

Specificity indicates that hybridisation to the target sequence generates a specific signal which can be distinguished from the signal generated through non-specific hybridisation. Perfectly complementary sequences are preferred to design probes according to this invention. It should be understood, however, that a certain degree of mismatch may be tolerated, as long as the specific signal may be distinguished from non-specific hybridisation.

The invention also concerns the use of a nucleic acid probe as described above in a method of detecting the presence, type or stage of progression of a PD in a subject, or in a method of assessing the response of a subject to a PD treatment.

The invention also relates to a nucleic acid chip comprising a probe as defined above. Such chips may be produced in situ or by depositing clones, by technique known in the art, and typically comprise an array of nucleic acids displayed on a matrix, such as a (glass, polymer, metal, etc.) slide.

An alteration in the BDKRB2 gene expression may also be detected by detecting the presence or (relative) amount of a BDKRB2 polypeptide. In this regard, a specific embodiment of this invention comprises contacting the sample (which may comprise biological fluids such as blood, plasma, serum, etc.) with a ligand specific for a BDKRB2 polypeptide, and determining the formation of a complex.

Different types of ligands may be used, such as specific antibodies. In a specific embodiment, the sample is contacted with an antibody specific for a BDKRB2 polypeptide, and the formation of an immune complex is determined. Various methods for detecting an immune complex can be used, such as ELISA, radioimmunoassays (RIA) and immuno-enzymatic assays (IEMA).

Further aspects and advantages of this invention will be disclosed in the following examples, which should be regarded as illustrative and not limiting the scope of this application. All cited publications or applications are incorporated therein by reference in their entirety.

### EXAMPLES

### A—Material and Methods

### Tissue Sources:

Appropriate patient samples were procured for evaluation of the research protocol. Samples were provided with relevant clinical parameters, and patient consent. Histological assessment was performed on all samples and diagnosis by pathology confirmed the presence and/or absence of Parkinson's disease (PD) within each sample. Clinical data generally included patent history, physiopathology, and parameters relating to PD physiology. Two or three normal and two or three PD substantia nigra and putamen samples were procured along with available clinical information. In addition, a universal RNA was obtained from a known commercial source, which was derived by pooling total RNA from ten different normal cell lines.

### SpliceArray™ Analysis:

Indirect comparisons were performed on the GPCR SpliceArray™ to look for alternative splicing events deregulated in Parkinson's disease (PD). Pools were made from substantia nigra and putamen tissue obtained postmortem from normal or PD patients. Each patient tissue pool was compared against a universal RNA pool on a GPCR SpliceArray™ and a dye swap was performed to determine any dye bias in the resulting data. Indirect log ration calculations were performed on the resulting data to determine the expression fold change between normal and PD samples for each probe on the array. The results were then prioritized based on fold change and p value. For a result to be considered significant it must have a fold change of greater than 2.0 and a p value less than 0.001.

### Expression Validation by RT-PCR:

Assessment of the expression profile for each prioritized SpliceArray™ probe was performed by RT-PCR or SYBR green RT-QPCR, procedures well known in the art. A protocol known as touchdown PCR was used, described in the user's manual for the GeneAmp PCR system 9700, Applied Biosystems. Briefly, PCR primers were designed to the monitored event and used for end point RT-PCR analysis or QPCR. Each RT reaction contained 5 μg of total RNA and was performed in a 100 μl volume using Archive RT Kit (Applied Biosystems). The RT reactions were diluted 1:50 with water and 4 μl of the diluted stock was used in a 50 μl PCR reaction consisting of one cycle at 94° C. for 3 min, 5 cycles at 94° C. for 30 seconds, 60° C. for 30 seconds and 72° C. for 45 seconds, with each cycle reducing the annealing temperature by 0.5 degree. This was followed by 30 cycles at 94° C. for 30 seconds, 55° C. for 30 seconds, and 72° C. for 45 seconds. 15 μl was removed from each reaction for analysis and the reactions were allowed to proceed for an additional 10 cycles. This produced reactions for analysis at 30 and 40 cycles, and allowed the detection of differences in expression where the 40 cycle reactions had saturated. Alternatively, a SYBR green QPCR master mix, Applied Biosystems, was used in a QPCR reaction, run on an ABI 7900HT, following the manufacturers suggested conditions and cycling parameters. The expression profile of each event was determined in normal and PD total RNA samples. Expression profiles were compared back to the SpliceArray™ results and those that correlated were considered validated expression results.

### Verification of RNA Structure:

SpliceArray™ identification of splice events that are altered between the experimental samples. However, the exact full coding sequence of the alternative isoform can not be determined directly from the SpliceArray™ data. The monitored event was used, however, to design experiments that elucidate the sequence of each transcript present in each sample. Primers were designed to amplify a region of the gene containing the monitored event and predicted coding region. These amplicons were subsequently cloned and sequenced for the identification of the exact exonic structure of each alternative isoform. This required cloning of the isoforms from an identified sample to verify the primary structure (sequence) of the isoforms. All original samples initially used to hybridize the SpliceArrays™ were used for the verification of the mRNA structure of the prioritized genes.

### Isolation of Full-Length Clones of Isoforms:

Isolation of the full-length clones containing all isoforms of interest was accomplished utilizing the information and DNA fragments generated during the structure validation process. Several methods are applicable to isolation of the full length clone. Where full sequence information regarding the coding sequence is available, gene specific primers were designed from the sequence and used to amplify the coding sequence directly from the total RNA of the tissue samples. An RT-PCR reaction was set up using these gene specific primers. The RT reaction was performed as described infra, using oligo dT to prime for cDNA. Second strand was produced by standard methods to produce double stranded cDNA. PCR amplification of the gene was accomplished using gene specific primers. PCR consisted of 30 cycles at 94° C. for 30 seconds, 55° C. for 30 seconds, and 72° C. for 45 seconds. The reaction products were analyzed on 1% agarose gels and the amplicons were ligated into prepared vectors with A overhangs for amplicon cloning. 1 μl of the ligation mixture was used to transform E. coli for cloning and isolation of the amplicon. Once purified, the plasmid containing the amplicon was sequenced on an ABI 3100 automated sequencer.

### Cell Based Rotenone Oxidative Stress Model System:

Neuroblastoma cells were treated with either 5 nm or 100 nm rotenone for 4 weeks or 48 hours, respectively, to induce oxidative stress. Rotenone is a known mitochondrial complex I inhibitor and this type of system has been shown to cause alpha-synucleic accumulation and a reduction in ATP levels (4). These treatments were used in the present study to monitor and look for expression correlations between this cell based system and the expression validated events described above for the patient samples. In many instances, using both standard RT-PCR and RT-QPCR, described above, we were able to see a similar deregulation of an identified event to what was observed in the PD patient samples. This led us to also use the rotenone model as a system to test prospective BDKRB2 agonists for protective effects against oxidative stress.

### MPTP Mouse Model of Parkinson's Disease:

Male mice were treated by intraperitoneal injection with either MPTP (45 mg/kg) or saline and selected tissues were harvested at 1, 3, 7, and 14 days post treatment. Saline treated control animals were only harvested for the 1 day time-point. RNA was isolated from the striatum of MPTP and saline treated animals and profiled by RT-QPCR, described above. We were able to detect the same down regulation of the BDKRB2 reference form observed in the PD patient samples and the rotenone treated SH-SY5Y cell model system, described above.

Monitoring GPCR Dependent Signaling with a Luciferase Reporter Assay:

The Promega Dual-Glo™ Assay system was used to indirectly monitor BDKRB2 dependent signaling. The pNFAT—Firefly Luciferase (Stratagene) reporter construct was used with the Dual-Glo™ assay system following the manufacturer's suggested protocol. CHO-K1 cells were transfected with pNFAT—Firefly Luciferase reporter, BDKBR2-pcDNA3.1 (Invitrogen) and pGL4.73 [hRenilla Luciferase/SV40—Promega]. 18 hrs after transfection, cells were treated with solvent or bradykinin at indicated concentrations in 0.05% FBS medium for 1, 4, 8 or 24 hrs. The Firefly luciferase reporter activity and the renilla luciferase activity were measured sequentially. BDKRB2 activity was estimated by calculating the Ratios of Firefly and Renilla Luciferase Units (RLU). A significant increase in RLU was observed after 4 and 8 hours of treatment with bradykinin.

### Cell Viability Assay:

Effect of bradykinin on rotenone toxicity and cell viability in SH-SY5Y cells. SH-SY5Y cells were pretreated with bradykinin (0 to 10 uM) in medium containing 10% FBS for 4 hours, then treated with 100 nM rotenone for 40 hours. The medium was replaced with 0.05% FBS medium without changing the bradykinin and rotenone concentrations for an additional 8 hrs. ATP levels were measured using a ATPlite™ (Promega) luminescence-based assay and the relative Counts Per Second (CPS) were plotted against bradykinin concentration. An increase in ATP levels which correlates to an increase in cell viability was observed in cells treated with bradykinin (300 nM and higher) over that observed in cells treated with Rotenone alone.

### B—Results

### Identification of BDKRB2 Isoforms:

Using methods described above, 2 alternative isoforms of the B₂ bradykinin receptor have been identified that are expressed and deregulated in Parkinson's disease patient tissue and model systems.

These DNA sequences are contained in Table 1 and correspond to the nucleic acid sequences of SEQ ID NOs: 1 and 2. Genomic sequence locations were generated using BLAT (Kent 2002) and the UCSC Genome Browser (Kent et al. 2002a) referencing the March 2006 human genomic assembly. The protein sequences encoded by these alternative isoforms are also contained in Table 1 and correspond to the amino acid sequences of SEQ ID NOs: 3 and 4, respectively.

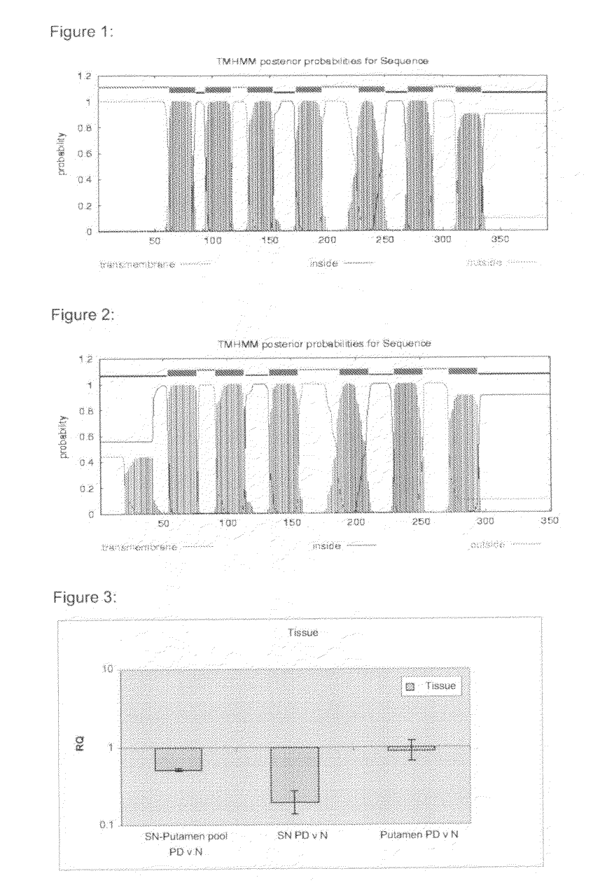

The variant isoform represented by SEQ ID NO: 2 contains a 120 base pair deletion of exon 3 of the reference form represented by SEQ ID NO: 1. This partial internal exon deletion event results in an in frame 40 amino acid deletion from the N-terminus of the protein. TMHMM (CBS) analysis reveals that this deletion event disrupts the N-terminal extracellular domain critical for ligand binding and the first transmembrane domain (compare FIG. 1 and FIG. 2). It is likely that the alternative BDKRB2 described by SEQ ID NO: 2 and 4 will have diminished signaling activity in vivo.

### RT-QPCR Analysis

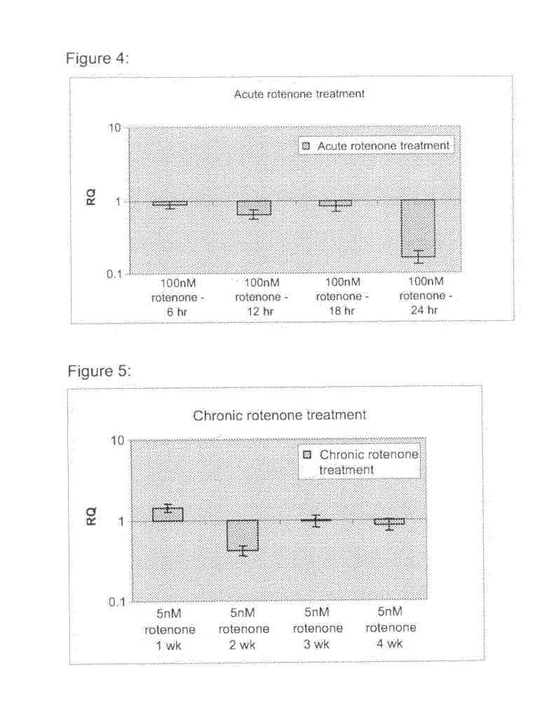

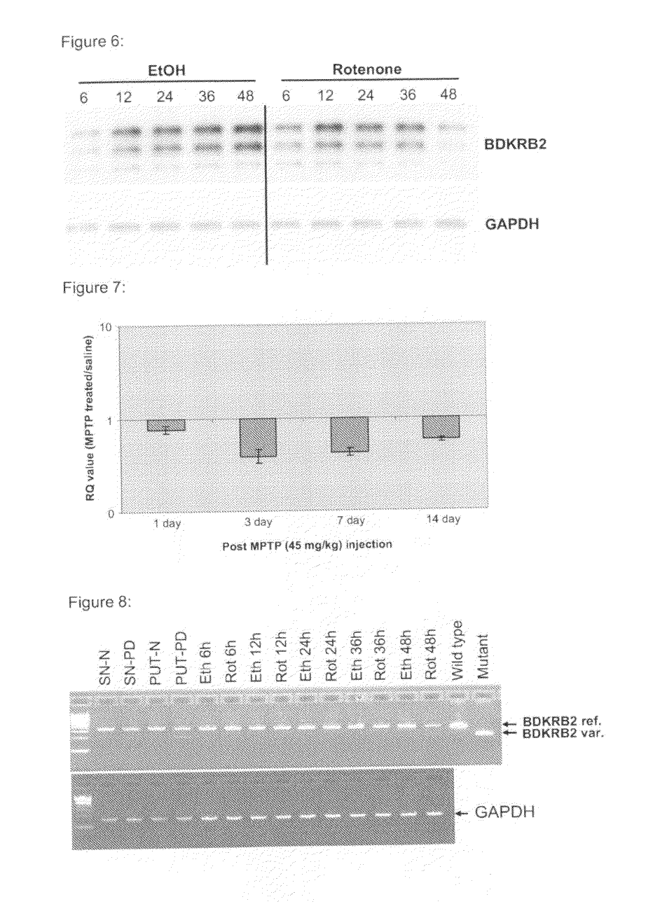

Similar to the SpliceArray™ results, which showed a 2.3 fold down regulation, RT-QPCR data demonstrates that the reference form of BDKRB2 is down regulated, and more specifically, down regulated only in PD substantia nigra tissue (FIG. 3). We were also able to show at the RNA level that the reference form of BDKRB2 is also down regulated in both acute and chronic rotenone model systems after 24 hours and 2 weeks of treatment respectively (FIGS. 4 and 5). In the acute rotenone system, bands cross reacting with an anti-BDKRB2 antibody were down regulated by 48 hours of exposure to 100 nm rotenone (FIG. 6). The reference form of BDKRB2 was also observed by QPCR to be down regulated in the striatum of MPTP treated mice (FIG. 7), which demonstrates consistent down regulation of the reference form of BDKRB2 in PD patient samples, animal models, and cell based systems.

### RT-PCR Analysis

Additionally, we were able to show by standard RT-PCR that the alternative isoform described here is present in PD patient samples and is upregulated as the reference form of BDKRB2 is down regulated in the acute rotenone model system, indicating an isoform switch from the reference form to the potentially less active variant form (FIG. 8).

### BDKRB2 Signaling Activity

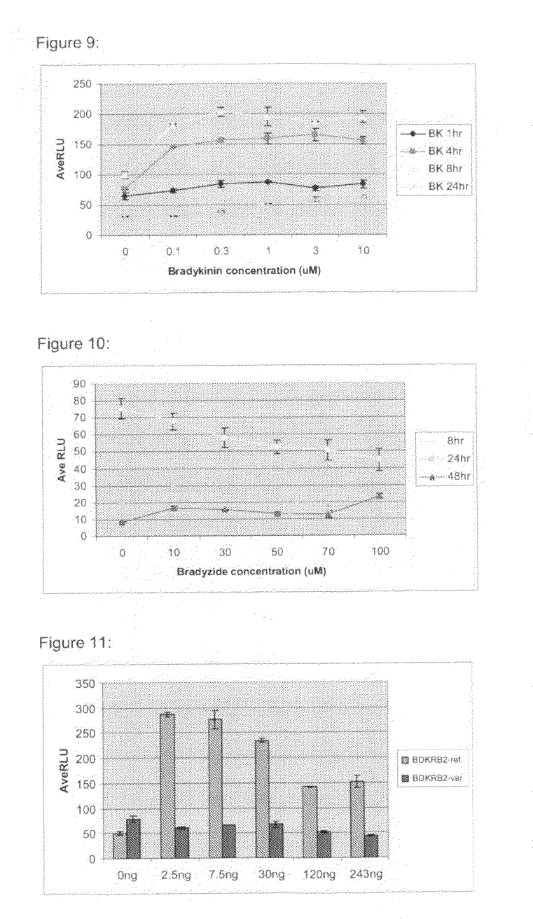

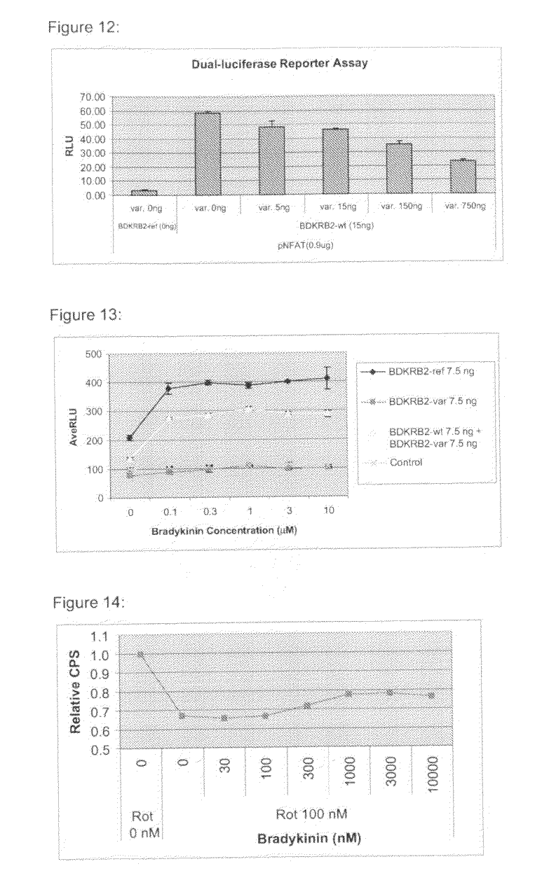

To determine time-points of greatest BDKRB2 signaling activity, a luciferase reporter assay was performed on extracts collected from CHO-K1 cells transiently expressing the BDKRB2 reference form. Significant luciferase activity was detected 4 hours after bradykinin treatment with the greatest activity being observed at 8 hours (FIG. 9). We also observed a significant serum induced activity in this assay at the 8 hour time-point, which could be inhibited by the BDKRB2 antagonist bradyzide (FIG. 10).

To compare the potential activity of the BDKRB2 reference and variant forms, the same luciferase reporter assay was used to record the respective activities of the two isoforms in the presence of 200 nM bradykinin (FIG. 11). Significant signaling activity was observed from the BDKRB2 reference form between 2.5 and 30 ng of transfected expression construct with decreased signaling at high concentrations. This is in stark contrast to the absolute lack of activity observed for the BDKRB2 variant.

Next we tested the effect on signaling of co-expressing the BDKRB2 reference and variant forms. Titrating in increasing concentrations of the variant form on top of a constant 15 ng of the reference form caused a dose dependent decrease in BDKRB2 dependent signaling (FIG. 13). The inhibitory effect of the variant form on signaling activity could not be overcome by titrating in increasing amounts of bradykinin (FIG. 13).

### Activity of BDKRB2 Agonists

Since all of the data indicates that BDKRB2 dependent signaling is decreased in Parkinson's disease due to down regulation of the reference form of the receptor and upregulation of an inhibitory variant; we tested the effect of bradykinin on cell viability in the acute rotenone cell model. An increase in relative cell viability was observed in cells treated with bradykinin (300 nm and higher) over that observed in cells treated with rotenone alone (FIG. 14).

TABLE 1
Sequence information for BDKRB2 alternatively spliced isoforms.

Accession #: NM_000623.2
Genomic sequence: chr14:95, 740, 950-95, 780, 536
Sequence definition: Homo sapiens bradykinin receptor B2 (BDKRB2)

Sequence ID NO: 1

CTCCGAGGAGGGGTGGGGACGGTCCTGACGGTGGGGACATCAGGCTGCCCCGCAGTACCAGGGAGCGACT
TGAAGTGCCCATGCCGCTTGCTCCGGGAGAAGCCCAGGTGTGGCCTCACTCACATCCCACTCTGAGTCCA
AATGTTCTCTCCCTGGAAGATATCAATGTTTCTGTCTGTTCGTGAGGACTCCGTGCCCACCACGGCCTCT
TTCAGCGCCGACATGCTCAATGTCACCTTGCAAGGGCCCACTCTTAACGGGACCTTTGCCCAGAGCAAAT
GCCCCCAAGTGGAGTGGCTGGGCTGGCTCAACACCATCCAGCCCCCCTTCCTCTGGGTGCTGTTCGTGCT
GGCCACCCTAGAGAACATCTTTGTCCTCAGCGTCTTCTGCCTGCACAAGAGCAGCTGCACGGTGGCAGAG
ATCTACCTGGGGAACCTGGCCGCAGCAGACCTGATCCTGGCCTGCGGGCTGCCCTTCTGGGCCATCACCA
TCTCCAACAACTTCGACTGGCTCTTTGGGGAGACGCTCTGCCGCGTGGTGAATGCCATTATCTCCATGAA
CCTGTACAGCAGCATCTGTTTCCTGATGCTGGTGAGCATCGACCGCTACCTGGCCCTGGTGAAAACCATG
TCCATGGGCCGGATGCGCGGCGTGCGCTGGGCCAAGCTCTACAGCTTGGTGATCTGGGGGTGTACGCTGC
TCCTGAGCTCACCCATGCTGGTGTTCCGGACCATGAAGGAGTACAGCGATGAGGGCCACAACGTCACCGC
TTGTGTCATCAGCTACCCATCCCTCATCTGGGAAGTGTTCACCAACATGCTCCTGAATGTCGTGGGCTTC
CTGCTGCCCCTGAGTGTCATCACCTTCTGCACGATGCAGATCATGCAGGTGCTGCGGAACAACGAGATGC
AGAAGTTCAAGGAGATCCAGACGGAGAGGAGGGCCACGGTGCTAGTCCTGGTTGTGCTGCTGCTATTCAT
CATCTGCTGGCTGCCCTTCCAGATCAGCACCTTCCTGGATACGCTGCATCGCCTCGGCATCCTCTCCAGC
TGCCAGGACGAGCGCATCATCGATGTAATCACACAGATCGCCTCCTTCATGGCCTACAGCAACAGCTGCC
TCAACCCACTGGTGTACGTGATCGTGGGCAAGCGCTTCCGAAAGAAGTCTTGGGAGGTGTACCAGGGAGT
GTGCCAGAAAGGGGGCTGCAGGTCAGAACCCATTCAGATGGAGAACTCCATGGGCACACTGCGGACCTCC
ATCTCCGTGGAACGCCAGATTCACAAACTGCAGGACTGGGCAGGGAGCAGACAGTGAGCAAACGCCAGCA
GGGCTGCTGTGAATTTGTGTAAGGATTGAGGGACAGTTGCTTTTCAGCATGGGCCCAGGAATGCCAAGGA
GACATCTATGCACGACCTTGGGAAATGAGTTGATGTCTCCGGTAAAACACCGGAGACTAATTCCTGCCCT
GCCCAATTTTGCAGGGAGCATGGCTGTGAGGATGGGGTGAACTCACGCACAGCCAAGGACTCCAAAATCA
CAACAGCATTACTGTTCTTATTTGCTGCCACACCTGAGCCAGCCTGCTCCTTCCCAGGAGTGGAGGAGGC
CTGGGGGCAGGGAGAGGAGTGACTGAGCTTCCCTCCCGTGTGTTCTCCGTCCCTGCCCCAGCAAGACAAC
TTAGATCTCCAGGAGAACTGCCATCCAGCTTTGGTGCAATGGCTGAGTGCACAAGTGAGTTGTTGCCCTG
GGTTTCTTTAATCTATTCAGCTAGAACTTTGAAGGACAATTTCTTGCATTAATAAAGGTTAAGCCCTGAG
GGGTCCCTGATAACAACCTGGAGACCAGGATTTTATGGCTCCCCTCACTGATGGACAAGGAGGTCTGTGC
CAAAGAAGAATCCAATAAGCACATATTGAGCACTTGCTGTATATGCAGTATTGAGCACTGTAGGCAAGAG
GGAAGAAAGAGAAGGAGCCATCTCCATCTTGAAGGAACTCAAAGACTCAAGTGGGAACGACTGGGCACTG
CCACCACCAGAAAGCTGTTCGACGAGACGGTCGAGCAGGGTGCTGTGGGTGATATGGACAGCAGAAGGGG
GAGACCAAGGTTCCAGCTCAACCAATAACTATTGCACAACCACCTGTCCCTGCCTCAGTTCCCTCTTCTG
TAACATGAAGTCGTTGTGAGGGTTAAAGGCAGTAACAGGTATAAAGTACTTAGAAAAGCAAAGGGTGCTA
CGTACATGTGAGGCATCATTACGCAGACGTAACTGGGATATGTTTACTATAAGGAAAAGACACTGAGGTC
TAGAAATAGCTCCGTGGAGCAGAATCAGTATTGGGAGCCGGTGGCGGTGTGAAGCACCAGTGTCTGGCAC
ACAGTAGGTGCTCATTGGCTCCCTTCCACCTGTCATTCCCACCACCCTGAGGCCCCAACCGCCACACACA
CAGGAGCATTTGGAGAGAAGGCCATGTCTTCAAAGTCTGATTTGTGATGAGGCAGAGGAAGATATTTCTA
ATCGGTCTTGCCCACAGGATCACAGTGCTGAGACCCCCCACCACCAGCCGGTACCTGGGAAGGGGGAGAG
TGCAGGCCTGCTCAGGGACTGTTCCTGTCTCAGCAACCAAGGGATTGTTCCTGTCAATCAATGGTTTATT
GGAAGGTGGCCCAGTATGAGCCCTAGAAGAGTGTGAAAAGGAATGGCAATGGTGTTCACCATCGGCAGTG
CCAGGGCAGCACTCATTCACTTGATAAATGAATATTTATTAGCTGGTTGGAGAGCTAGAACCTGGAGAGG
CTAGAACCTGGAGAACTAGAACCTGGAGGGCTAGAACCTGGAGAGGCTAGAACCAAGAAGGGCTAGAACC
TGGAGGGGCTAGAACCTAGAGAAGCTAAAACCTGAGCTAGAAGCTGGAGGACTAGAACCTGGAGGGCTGG
AATCTGGAGAGCTAGAACCTGGAGGGCTAGAACCTGGAGGGCTAGAATCTGGAGAGCTAGAACCTGGAGG
GCTAGAATCTGGAGAGCTAGAACCTGGAGGGCTAGAACCTGGAGAGCTAGAACCTAGAAGGGCTAGAACC
TGGAGGGCTGGAATCTGGAGAGCTAGAACCTGGAGGGCTAGAACCTGGAGGGCTAGAACCTAGAAGGGCT
AGAACCTGGAGGGCTGGAATCTGGAGAGCTAGAACCTGGAGGGCTAGAACCTGGAGGGCTAGAACCTAGA
AGGGCTAGACCTGGAGGGCTAGAAACCTGGCAGGTTAGAACCTAGAAGGGCTAGAACCTGGAGAGCCAGA
ACCTGGAGGGCTAGAACCTGGAAGGGCTAGAACCTAGAGAGCTAGAACATGGAGAGCTAGAACCCGGCAG
GCTAGAACCTGGCAAGCTAGAACCTGGAGGGAATGAACCTGGAGGGCTAGAACCTGGAGAATGAGAAAAA
TTTACATGGCAAAGAGCCCATAAATCCTGACCAATCCAACTCTGAATTTTAAAGCAAAAGCGTCAAAAAA
AAGATTCCCTCCTTACCCCCAACCCACTCTTTTTTCCCACCACCCACTCTCCTCTGCCTCAGTAAGTATC
TGGAGGAAGAAAACAGGTGAAAGAAGAACTAAAAACCATTTAGTATTAGTATTAGAATGAAGTCAAACTG
TGCCACACATGCTGAATGAAAAAAAAAAAAAGAGGCTGTGTTTTGTCACACAGGGCAGTCATTCAGCACC
AGAGCACGTGATGGTCTGAGACTCTCTTAGGAGCAGAGCTCTGCCGCAATGGCCATGTGGGGATCCACAC
CTGGTCTGAGGGGCAACTGAGTCTGCGGGAGAAGAGCGGCCCTATGCATGGTGTAGATGCCCTGATAAAC
AACATCTGTCCTGTGAAAGACTCAATGAGCTGTTATGTTGTAAACAGGAAGCATTTCACATCCAAACGAG
AAAATCATGTAAACATGTGTCTTTTCTGTAGAGCATAATAAATGGATGAGGTTTTTGCATAGCTCTAGCA
TTTGTTACAACTCCCGAAACCCCCGAGTTTGGTCCCTGGGGTACCGCCTTGCACACTCAGAAGCCTTTGG
GAAGGGGTGCTATTCATTTCTGCTCAATCTGTTAACAGGCTTCTGGCATGTAGATCAGTGGTCTCCAAGC
TTTTGTGATTGTATATTCCTATAGGAAAAAAAGAATTGATTATGCATACCCAGTATGTATACTTATTAAT
CTGTATGAAGATGTACATTCTAAAATATAATCAACCAGTAGAAATTTAAGAAAGAAGATGTAAAAAA
Accession #: N/A
Genomic sequence: chr14:95, 740, 950-95, 780, 536
Sequence definition: Homo sapiens bradykinin receptor B2 (BDKRB2)
variant

Sequence ID NO: 2

CATCCCACTCTGAGTCCAAATGTTCTCTCCCTGGAAGATATCAATGTTTCTGTCTGTTCGTGAGGACTCC
GTGCCCACCACGGCCTCTTTCAGCGCCGACATGCTCAATGCCACCCTAGAGAACATCTTTGTCCTCAGCG
TCTTCTGCCTGCACAAGAGCAGCTGCACGGTGGCAGAGATCTACCTGGGGAACCTGGCCGCAGCAGACCT
GATCCTGGCCTGCGGGCTGCCCTTCTGGGCCATCACCATCTCCAACAACTTCGACTGGCTCTTTGGGGAG
ACGCTCTGCCGCGTGGTGAATGCCATTATCTCCATGAACCTGTACAGCAGCATCTGTTTCCTGATGCTGG
TGAGCATCGACCGCTACCTGGCCCTGGTGAAAACCATGTCCATGGGCCGGATGCGCGGCGTGCGCTGGGC
CAAGCTCTACAGCTTGGTGATCTGGGGGTGTACGCTGCTCCTGAGCTCACCCATGCTGGTGTTCCGGACC
ATGAAGGAGTACAGCGATGAGGGCCACAAOGTCACCGCTTGTGTCATCAGCTACCCATCCCTCATCTGGG
AAGTGTTCACCAACATGCTCCTGAATGTCGTGGGCTTCCTGCTGCCCCTGAGTGTCATCACCTTCTGCAC
GATGCAGATCATGCAGGTGCTGCGGAACAACGAGATGCAGAAGTTCAAGGAGATCCAGACaGAGAGGAGG
GCCACGGTGCTAGTCCTGGTTGTGCTGCTGCTATTCATCATCTGCTGGCTGCCCTTCCAGATCAGCACCT
TCCTGGATACGCTGCATCGCCTCGGCATCCTCTCCAGCTGCCAGGACGAGCGCATCATCGATGTAATCAC
ACAGATCGCCTCCTTCATGGCCTACAGCAACAGCTGCCTCAACCCACTGGTGTACGTGATCGTGGGCAAG
CGCTTCCGAAAGAAGTCTTGGGAGGTGTACCAGGGAGTGTGCCAGAAAGGGGGCTGCAGGTCAGAACCCA
TTCAGATGGAGAACTCCATGGGCACACTGCGGACCTCCATCTCCGTGGAACGCCAGATTCACAAACTGCA
GGACTGGGCAGGGAGCAGACAGTGAGCAAA
Accession #: NP_000614.1
Sequence definition: Homo sapiens bradykinin receptor B2 (BDKRB2)

Sequence ID NO: 3

MFSPWKISMFLSVREDSVPTTASFSADMLNpVTLQGPTLNGTFAQSKCPQVEWLGWLNTIQPPFLWVLFVL
ATLENIFVLSVFCLHKSSCTVAEIYLGNLAAADLILACGLPFWAITISNNFDWLFGETLCRVVNAIISMN
LYSSICFLMLVSIDRYLALVKTMSMGRMRGVRWAKLYSLVIWGCTLLLSSPMLVFRTMKEYSDEGHNVTA
CVISYPSLIWEVFTNMLLNVVGFLLPLSVITFCTMQIMQVLRNNEMQKFKEIQTERRATVLVLVVLLLFI
ICWLPFQISTFLDTLHRLGILSSCQDERIIDVITQIASFMAYSNSCLNPLVYVIVGKRFRKKSWEVYQGV
CQKGGCRSEPIQMENSMGTLRTSISVERQIHKLQDWAGSRQ
Accession #: N/A
Sequence definition: Homo sapiens bradykinin receptor B2 (BDKRB2)
variant

Sequence ID NO: 4

MFSPWKISMFLSVREDSVPTTASFSADMLNATLENIFVLSVFCLHKSSCTVAEIYLGNLAAADLILACGL
PFWAITISNNFDWLFGETLCRVVNAIISMNLYSSICFLMLVSIDRYLALVKTMSMGRMRGVRWAKLYSLV
IWGCTLLLSSPMLVFRTMKEYSDEGHNVTACVISYPSLIWEVFTNMLLNVVGFLLPLSVITFCTMQIMQV
LRNNEMQKFKEIQTERRATVLVLVVLLLFIICWLPFQISTFLDTLHRLGILSSCQDERIIDVITQIASFM
AYSNSCLNPLVYVIVGKRFRKKSWEVYQGVCQKGGCRSEPIQMENSMGTLRTSISVERQIHKLQDWAGSR
Q

### REFERENCES

Anderson. Curr Treat Options Neurol. 6(3):201-207, 2004
Burch and Axelrod. Proc Natl Acad Sci USA. 84:6374-6378, 1987
deBolis et al. Immunopharmacology. 17:187-198, 1989
Emerich et al. Clinical Pharmacokinetics. 40(2):105-123, 2001 EP1,062,364
Floyd and Hensley. Neurobiol Aging. 23:795-807, 2002
Jouaville et al. Proc Nat Acad Sci USA. 96:13807-13812, 1999
Kato et al. FEBS Lett. 232:252, 1988
Kaya et al. J Biol Chem. 264:4972-4977, 1989
Kent. Genome Res. 12(4):656-64, 2002
Kent et al. Genome Res. 12:996-1006, 2002a
Kidd. Altern Med Rev. 10(4):268-293, 2005
Kidd. Altem Med Rev. 5:502-529, 2000
Louis et al. Arch Neurol. 61(8):1273-6, 2004
Olanow and Tatton. Annual Review of Neuroscience. 22: 123-144, 1999
Perkins and Kelly. Br J Parmacol. 110:1441-1444, 1993
Regoli et al. Can J Physiol Pharmacol. 56:674-677, 1978
Rizzi et al. Naunyn Schmiedebergs Arch Pharmacol. 360(4):361-7, 1999
Slivka and Insel. J Biol Chem. 263:14640-14647, 1988
Tabner et al. Curr Top Med Chem. 1(6):507-17, 2001
Toescu and Verkhratsky. Cell Calcium. 34:311-323, 2003
U.S. Pat. No. 5,874,442
U.S. Pat. No. 6,881,571
Visch et al. J Biol Chem. 279(39):40328-40336, 2004
WO01/46137
WO97/17070
WO97/30992
WO98/00409

1. An isolated nucleic acid molecule that is expressed by human Parkinson's disease cells, selected from the group consisting of:
(i) a nucleic acid comprising the sequence contained in SEQ ID NO: 1 or 2;
(ii) variants of (i), wherein such variants comprise a nucleic acid sequence that is at least 70% identical to the sequence of (i) when aligned without allowing for gaps; and
(iii) fragments of (i) or (ii) having a size of at least 20 nucleotides in length.

2. The nucleic acid molecule of claim 1, which comprises the nucleic acid sequence of SEQ ID NO: 1 or 2 or a fragment thereof.

3. A primer mixture that comprises primers that result in the specific amplification of one of the nucleic acid sequences of claim 1.

4. A polypeptide comprising the amino acid sequence of SEQ ID NO: 3 or 4 or a fragment thereof.

5. A diagnostic kit for detection of Parkinson's disease which comprises a nucleic acid according to claim 1 and a detectable label.

6. A method for treating Parkinson's disease, which comprises administering to a subject a therapeutically effective amount of a ligand which specifically binds a target molecule selected from a nucleic acid molecule of claim 1.

7. The method of claim 6 wherein said ligand is a monoclonal antibody or fragment thereof.

8. The method of claim 6 wherein said ligand is a small molecule.

9. The method of claim 6 wherein said ligand is a peptide.

10. The method of claim 6, wherein said ligand binds an extracellular domain of said polypeptide.

11. The use of a BDKRB2 agonist for the manufacture of a medicament for treating Parkinson's Disease.

12. The use of claim 11, for the manufacture of a medicament for protecting neurons from oxidative stress in a subject having Parkinson's Disease.

13. The use of claim 11, for the manufacture of a medicament for protecting dopaminergic neurons in a subject having Parkinson's Disease.

14. The use of claim 11, wherein the agonist is a compound having an IC50 for BDKRB2 that is below about 1 mM, preferably below 50 nM.

15. The use of claim 11, wherein the agonist is selective for BDKRB2.

16. The use of claim 11, wherein the agonist crosses the blood-brain barrier.

17. The use of claim 11, wherein the agonist is a compound having a molecular weight below about 800 daltons.

18. The use of claim 11, wherein the agonist is a compound selected from:
Bradykinin: Arg-Pro-Pro-Gly-Phe-Ser-Pro-Phe-Arg-OH;
[Hyp3]-Bradykinin: Arg-Pro-Hyp-Gly-Phe-Ser-Pro-Phe-Arg;
FR 190997 and
Labradimil;
as well as their optical and geometrical isomers, racemates, tautomers, salts, hydrates and mixtures thereof.

19. The use of claim 11, wherein the agonist is formulated in any pharmaceutically acceptable carrier(s) or excipient(s).

20. The use of claim 19, wherein the agonist is incorporated into a specific pharmaceutical formulation or technology allowing delivery to the human brain using catalyzed-transport systems.

21. The use of claim 20, wherein said formulation or technology is selected from liposomal carriers and nanoparticles.

22. The use of claim 11, wherein the agonist is administered to said subject by systemic injection(s) or oral administration(s).

23. The use of claim 11, wherein a combination of BDKRB2 and BDKRB1 agonists is administered.

24. The use of claim 11, wherein the agonist(s) is administered in combination with another active agent.
